(19) United States
(12) Patent Application Publication
(10) Pub. No.: US 2004/0171692 A1
(43) Pub. Date: Sep. 2, 2004

(54) Modulation of bone formation

(76) Inventors: Scutt Andrew, Sheffield (GB); Still Karen, Sheffield (GB)

Correspondence Address: FISH & RICHARDSON PC, 225 FRANKLIN ST, BOSTON, MA, 02110, US

(21) Appl. No.: 10/375,968

(22) Filed: Feb. 28, 2003

(51) Int. Cl. A61K 31/192; A61K 31/198

(52) U.S. Cl. 514/563; 514/571

(57) ABSTRACT

The use of an activator or ligand of a peroxisome proliferator-activated receptor, other than PPARγ, or pharmaceutically acceptable derivative of said activator or ligand, in the manufacture of a medicament for the treatment or prophylaxis of bone disease allows, for the first time, bone anabolism to enhance the deposition of bone in conditions which would benefit from increased bone deposition. The reverse, where there is inhibition and/or retardation of bone deposition is also facilitated.

The present invention relates to the use of agents which modulate the activity of peroxisome proliferator-activated receptors (PPAR's), in therapy, and to assays for such agents.

The mammalian skeleton provides a number of functions, such as the provision of support, the protection of internal organs and the provision of sites for the attachment of muscles and tendons which operatively function to enable an animal to move. Bone is a living tissue which is being constantly resorbed, replaced and remodelled during growth and development. This is particularly prevalent during the developmental stages of the mammal when the growth of the skeleton has to be co-ordinated with the growth and development of the mammal's various organ systems. When the adult skeleton is formed it requires constant maintenance to ensure its functions are adequately maintained.

The deposition, resorption and/or remodelling of bone tissue is undertaken by specialised, anabolic cells known as osteoblasts (involved in bone tissue deposition) and catabolic cells, known as osteoclasts (involved in the resorption and/or remodelling of bone tissue). The activity of these specialised cells varies during growth and development. During normal, early human development, new bone tissue is formed faster than old bone is resorbed, resulting in bone becoming larger, heavier and more dense. In the fully developed human adult, peak bone density mass is achieved during the late 20's. However, in later life, osteoclast activity exceeds that of osteoblasts, resulting in a decrease in bone density and, consequently, a reduction in bone mass.

There is a number of conditions which result in abnormal bone formation and which can result in severe consequences during early development and/or in later life. Diseases of this type include, osteoporosis, osteopetrosis, hypophosphatasia, osteogenesis imperfecta, Paget's disease, deafness and hypercalcaemia as a result of cancer.

By far the most common one disorder is osteoporosis. Osteoporosis is a disease characterised by a thinning and loss of structural integrity of the bone tissue causing the skeleton to become susceptible to fracture, typically of the spine, wrist or hip. Up to 200 million people world-wide suffer from osteoporosis and every year 700,000 people in Europe, the USA and Japan suffer a hip fracture. Of these, 20% die within six months and 50% never return to a fully independent lifestyle.

Women are more prone to osteoporosis, but other contributory factors include, being thin and/or small, age, being occidental, and hereditary factors. In addition abnormal hormone levels (e.g. low oestrogen levels in females, low testosterone levels in males) and deficiencies of calcium and/or vitamin D may also contribute. These are uncontrollable, but controllable factors include; having a sedentary lifestyle, early menopause, anorexia nervosa or bulimia, amenorrhoea, certain therapeutic agents (e.g. corticosteroids, anticonvulsants), smoking and alcohol abuse. Prophylactic measures include; exercise, ensuring the provision of sufficient calcium in the diet, and the provision of vitamin D supplements.

Other substances have been shown to stimulate bone formation when administered to adult animals, and these include, parathyroid hormone (PTH), prostaglandin E₂ (PGE₂) and 1,25-(OH)₂-vitamin D₃ μ1,25-(OH)₂-D₃). However, these are all-associated with side effects limiting their clinical use. For example, PGE₂ has been associated with spontaneous abortion, diarrhoea and circulatory collapse; while 1,25-(OH)₂-D₃ may cause hypercalcaemia leading to kidney calcification; and PTH, which has to be administered by injection, can cause modest hypercalcaemia. Raloxifene and Alendronate are both useful, but are associated with side effects, including-hot flushes, deep vein thrombosis, abdominal or musculoskeletal pain, nausea, heartburn or irritation of the oesophagus.

Hormone replacement therapy (HRT) has been used for the treatment of post-menopausal osteoporosis, but both oestrogen and calcitonin (components of HRT) are associated with risks and/or side effects. Oestrogen may increase the risk of endometrial cancer, while calcitonin can cause flushing of the face and hands, increased urination, nausea, and skin rashes.

It is clear that existing therapies for the treatment of osteoporosis, although effective at promoting either bone deposition or inhibiting excessive bone resorption, have unacceptable side effects which restrict their clinical use. Further, effective therapies are still needed.

Another disease which results in abnormal bone formation is Paget's disease. This typically results in enlarged and deformed bones which can result in weakening of bones, resulting in increased fractures, bone pain and arthritis. A related symptom of Paget's disease is hearing loss. The causes of Paget's disease are much less clearly defined. Up to 40% of patients have a positive fairly history of the disorder, but data also support a viral aetiology for Paget's disease. As with osteoporosis, therapies to ameliorate the symptoms of Paget's disease include exercise, and the administration of calcitonin or bisphosphonates.

Hyperparathyroidism is a hormonal condition which can result in loss of bone, occurring when the parathyroid glands become overactive and produce too much parathyroid hormone. PTH promotes the release of calcium from bones and regulates the absorption of calcium from food. Symptoms associated with hyperparathyroidism include lethargy, fatigue, muscle weakness, joint pains and constipation, and the high serum levels of calcium can also result in calcium deposition in the kidneys, resulting in stones. The cause of this disease is at present unknown. Treatment is typically by the removal of one or more of the parathyroid glands, but this may lead to hypoparathyroidism which is irreversible and untreatable.

Osteogenesis imperfecta (OI) is a disease characterised by fragile bones, and results from an abnormal or reduced ability of bone tissue to produce collagen. The different types are of varying severity and effect, the mildest type being characterised by a predisposition to bone fracture, a tendency towards spinal curvature, brittle teeth and hearing loss. There is no known cure, and treatment is through physical therapy to minimise the symptoms and to reduce the likelihood of bone fracture. Promising results have been reported for bisphosphonates, particularly in growing children, but these trials have not yet been blinded or placebo controlled.

A related, genetically inherited disorder, referred to as hypophosphatasia, has many symptoms in common with 01. In severe cases of this disease the individuals fail to form a skeleton in the womb and are stillborn. In milder cases, for example odontohypophosphatasia, the disease is manifested by premature loss of teeth. There is no treatment for hypophosphatasia.

Other conditions can also have indirect consequences for bone formation. Of particular importance is cancer, which can result in hypercalcaemia and, consequently, fragile bones.

Peroxisome proliferator-activated receptors (PPAR's) are a group of hormone receptors, located in the nucleus, controlling the expression of genes involved in lipid homeostasis. PPAR's have been shown to respond to a number of compounds promoting the replication of peroxisomes and their capacity to metabolise fatty acids via increased expression of the enzymes contained within the peroxisomes.

PPARα was the first member of this family to be characterised [Isseman & Green (1991), Nature, 347: 645-650], and is activated by a number of medium and long-chain fatty acids which stimulate the expression of genes involved in peroxisomal β-oxidation. PPARα exerts its effect on lipid metabolism through upstream DNA enhancer elements and has been shown to form a heterodimer with the retinoid X receptor [Kliewer et al. (1992), Nature, 358: 771-774], which complex has been shown to bind the enhancer elements and to activate RNA polymerase II transcription.

Since the identification of PPARα, other members of the PPAR family have been identified, including PPARγ (Kliewer et al., Proc. Nat. Acad. Sci. USA, 91: 7355-7359) and PPARδ[Lim H., et al., (1999), Cyclo-oxygenase-2-derived prostacyclin mediates embryo implantation in the mouse via PPARδ]. Each of these PPAR homologues has been shown to bind a number of compounds capable of inducing peroxisome replication/activity via PPAR gene specific transcription.

Many of the agents shown to bind PPAR homologues have been shown to have potential in therapy. For example, WO 99/32465 describes arylthiazolidinedione derivatives which bind PPARα, δ and/or γ and which may be useful in the treatment or prevention of diabetes, hyperglycaemia, hyperlipidaemia, atherosclerosis, or obesity.

In addition to general PPAR agonists, a number of specific agents have been identified which are claimed to specifically activate particular PPAR transcription factors. For example WO 97/36579 discloses a PPARα agonist which has utility in the treatment of obesity. WO 97/28149 discloses compounds which are PPARδ agonists useful in raising high density lipoprotein plasma levels, thereby arresting the progression of atherosclerotic cardiovascular diseases. U.S. Pat. No. 5,925,657 discloses the use of a PPARγ agonist in the inhibition of cytokine production associated with an inflammatory response typically associated with rheumatoid arthritis.

WO 99/10532 discloses further methods to identify both PPAR agonists and PPAR antagonists to identify agents which may have use in regulating the activity of PPAR homologues.

EP-A-783888 discloses the use of troglitazone and related thiazolidinediones in the manufacture of medicaments for the treatment and prophylaxis of osteoporosis, although anabolic activity in bone tissue is not demonstrated.

WO 00/27832 is an intermediate document and discloses PPARγ antagonists which may be used in the treatment of osteoporosis.

WO 00/23451 is an intermediate document and discloses substituted, tricyclic compounds in the treatment and/or prevention of conditions mediated by PPAR's, particularly hypolipidaemia and diabetes.

JP-A-2022226 discloses the use of prostaglandin D and J analogues, in the treatment of bone diseases, by demonstrating positive effects on osteoblasts. There is no mention of any effect on PPAR.

WO 00/18234 is an intermediate document and discloses thiazolidinediones as PPARγ agonists in combination as therapeutic agents for tumour therapy. Tumours were reduced but no bone anabolic activity shown.

Okazaki et al. [Endocrinology, (1999), 140(11): 5060-5] report the involvement of a group of thiazolidinedione compounds (PPARγ agonists) which inhibit, in vitro, the formation of osteoclasts from bone marrow stromal cells (BMSC's). BMSC's exposed to thiazolidinediones induce the formation of adipocytes and inhibit osteoclast formation.

The involvement of PPAR agonists in the differentiation of adipocytes, at the expense of osteoblast formation, is described in Johnson et al. [Endocrinology (1999), 140(7), p3245]. Both osteoblasts and adipocytes originate from bone marrow mesenchymal stem cells, and enhancing the production of one inhibits production of the other. Glucocorticoid receptors have been shown to have pro-osteoblastic activity but PPAR ligands are here shown to promote adipocyte differentiation.

Johnson et al. describe the effects of TZD, [5-(4-{[N-methyl-N(2-pyridyl)-amino]ethoxy}benzyl)thiazolidine-2,4-dione] a PPARγ agonist, in combination with dexamethasone (a glucocorticoid) on MB-1.8 cells, an osteoblastic cell-line. MB-1.8 cells, when exposed to TZD, showed a decrease both in alkaline phosphatase activity and in the expression of osteoblast-associated genes, while enhancing the expression of adipocyte fatty acid protein. Dexamethasone counteracted the effects of TZD on alkaline phosphatase and osteoblast gene marker expression, but augmented the expression of adipocyte fatty acid protein. Thus, again, it is shown that PPAR agonists promote adipocyte differentiation at the expense of osteoblast differentiation.

Thus, it has been established that, while the main area of activity of the PPAR's is in lipid homeostasis, they can also have an effect on bone metabolism. This effect appears to be by affecting differentiation of stem cells in bone, and has only been shown to be negative, in that the activated PPAR favours the formation of adipocytes at the expense of osteoblasts. This is not at all surprising, given that PPAR's are active in lipid homeostasis, and is certainly true of the PPARγ agonists, such as proglitazone, for example, which have been studied to date.

It has also been shown that it is possible to reduce the numbers of osteoclasts, thereby slowing bone resorption but, by the time osteoporosis, for example, is diagnosed, the patient may already have lost 50% of bone mass, and there is a need, not for a static therapy, such as might be obtained by preventing production of further osteoclasts, but for a regenerative therapy.

Surprisingly, we have now found that PPARα and PPARδ are not only involved in lipid homeostasis, but also in the regulation of bone formation by osteoblasts and, when suitably activated, actually enhance osteoblastic activity.

Thus, in a first aspect, there is provided the use of an activator or ligand of a peroxisome proliferator-activated receptor other than PPARγ, or pharmaceutically acceptable derivative of said activator or ligand, in the manufacture of a medicament for the treatment or prophylaxis of bone disease.

In an alternative aspect, there is provided the use of at least one agent capable of modulating the activity of at least one PPAR transcription factor in the manufacture of a medicament for the treatment of at least one bone disorder.

The term “activator” is used, herein, to refer to substances which activate a PPAR. Such substances may activate the PPAR directly, or may be metabolised in vivo, to form a ligand to activate the PPAR by binding thereto.

It will be appreciated that certain substances are pan-activators, or pan-agonists, and can activate all PPAR's, and that these substances, per se, do not necessarily bind the receptor. Such substances are included within the scope of the present invention, provided that the osteoblastic activity resulting from the activated PPAR is greater than normal, preferably as determined by the test of the invention, described hereinunder. Preferred pan-agonists for use in the present invention include linoleic acid, linolenic acid and arachidonic acid.

It will be appreciated that pharmaceutically acceptable derivatives of the activators or ligands of the invention may be employed, as desired. Such derivatives may take the form of pro-drugs, salts or esters of the ligand or activator, and may be active in their own right. Preferred salts are simple salts, such as the chloride, sulphate, or acetate. Preferred esters include the ethyl and methyl esters, while suitable pro-drugs include the glycosides of the compounds.

It will be appreciated that there are at least three types of PPAR, namely PPARα, PPARγ and PPARδ. There may well be further receptors in this family, and these are also included within the scope of the present invention.

It will be appreciated that the compounds for use in the present invention are those which bind to, or activate, PPAR's and all are included in the present invention, provided that they bind or activate a PPAR other than, or in addition to, PPARγ.

It will also be appreciated that the present invention extends to novel compounds, as disclosed herein.

In a preferred embodiment, the compounds used are PPAR antagonists, and may be of use in the treatment of Paget's disease.

However, it is particularly preferred that the compounds for use in the present invention are agonists, or activators, of the PPAR's. Agonists for PPAR's other than PPARγ promote osteoblastic activity and are useful in the treatment of conditions in which the patient suffers from reduced, or insufficient, bone mass, such as osteoporosis. Previous treatments have only been static, but compounds of the present embodiment of the invention allow bone to be regenerated.

A preferred class of compounds is those which activate PPARα or PPARδ.

Also preferred are the fibrates. Some of the fibrates activate PPARγ, but fenofibrate is an agonist for PPARα and bezafibrate is an agonist for PPARδ. Either of these compounds, individually, is preferred.

It will be apparent to one skilled in the art that the term agonist refers to a general group of agents which are capable of promoting the activity of PPAR transcription factors. Accordingly, the use of the term antagonist refers to any agent capable of inhibiting the transcriptional activity of PPAR transcription factors.

In yet a further preferred embodiment of the invention the agonist is a fibrate or a N-(2-benzoylphenyl)-L-tyrosine derivative. Glitazones which only serve as PPARγ agonists are not a part of the present invention, and glitazones are only preferred when they serve as agonists or antagonists for other PPAR's.

The following agents are all, independently, preferred: 3-{4[2-(2-benzoxazolylmethylamino)ethoxy]benzene}-2-(2S)-(2,2,2-trifluoroethoxy)propanoic acid; docosahexaenoic acid; LY171883; linoleic acid; oleic acid; palmitic acid; clofibrate; eicosatetraenoic acid; 8(S)-hydroxy-6,8,11,14 eicosatetraenoic acid; methyl palmitate; Wy-14643 ([4-chloro-6-(2,3-xylidino)-2-pyrimidinylthio]acetic acid); nafenopin {2-methyl-2[p-(1,2,3,4-tetrahydro-1-naphthyl)phenoxy]propionic acid}; clofibric acid [2-([p]-chlorophenoxy)-2-methylpropionicacid]; MK-571 ((+-)-3-[({3-[2-(7-chloro-2 quinolinyl)ethenyl]-phenyl} {[3-(dimethylamino)-3-oxopropyl]thio}methyl)-(thio) (propanoic acid); PGJ(2)[prostaglandin J₂]; Δ(12)PGJ(2) [Δ(12)prostaglandin J₂]; 15-deoxy-Δ(12,14)-PGJ(2) [15-deoxy-Δ(12,14)-prostaglandin J₂]; PD19559; conjugated linoleic acid; carbaprostacyclin; 9-hydroxyoctadecadienoic acid; KRP-297; Iloprost; L783483; petroselinic acid; elaidic acid; erucic acids, linolenic acid; L165461; L796449; L165041; GW2433; GW1929; GW2331; 2 bromopalmitate; heptyl-4-yn-VPA (heptyl-4-yn-valproic acid); hexyl-4-yn-VPA (hexyl-4-yn-valproic acid); methyl palmitate; 4-[3-(2-propyl-3-hydroxy-4-acetylphenoxy)propyloxy]-phenoxyacetic acid; 3-chloro-4-{3-[2-propyl-3-hydroxy-4-(1-hydroxliminopropyl)-phenoxy]propylthio}phenylacetic acid; 3-chloro-4-[3-(3-ethyl-7-propyl-6-benz [4,5]-isoxazoloxy)propylthio]phenyl acetic acid; 3-chloro-4-[3-(2-propyl-3-trifluoromethyl-6-benz-[4,5]-isoxazoloxy)propylthio]phenyl acetic acid; 4-(2-acetyl-6-hydroxyundecyl)cinnamic acid; 3-chloro-4-[3-(3-phenyl-7-propylbenzofuran-6 yloxy)propylthio]phenylacetic acid; and 3-propyl-4-[3-(3-trifluoromethly-7-propyl-6-benz[4,5]-isoxazoloxy)propylthio]phenyl acetic acid.

Preferred targets for therapy are, individually: osteoporosis; Paget's disease; osteogenesis imperfecta; hypophosphatasia; hyperparathyroidism; deafness; orthodontic abnormalities; or cancers which result in hypercalcaemia, especially myeloma.

Osteoporosis targets are, preferably, post menopausal osteoporosis, male osteoporosis or hormonally induced osteoporosis, especially where induced by a glucocorticoid.

The invention further envisages a method for the treatment of a mammal, preferably a human, who is either susceptible to or has a bone disorder, comprising administering a pharmacologically effective amount of an activator or ligand of the present invention.

The present invention further provides pharmaceutical formulations of ligands and activators as described herein, especially where such have not previously been disclosed for therapeutic use.

It will be appreciated that therapeutic formulations may take any suitable form, and any pharmaceutically acceptable carrier or carriers may be used. These will depend on the nature of the compound(s) used in the formulation which may, in turn, be in the form of pro-drugs, salts or esters.

Suitable carriers may simply be water or saline, but it is generally preferred that the compounds be administered systemically. This may be by injection, time-release capsule/tablet, or transdermal patch, for example. Suitable formulations for all of these are well known in the art, and will be readily apparent to those skilled in the art.

In yet still a further preferred embodiment of the invention the medicament comprises at least one carrier and/or excipient. Ideally the carrier or excipient functions to modulate the stability and/or targeting of the agent to its preferred site of activity, generally bone tissue. Suitable carriers and/or excipients for targeting are well known in the art, and include antibodies specific to polypeptides differentially expressed by selected cell types; and liposomes, such as so called STEALTH® liposomes. Other suitable targeting substances may be incorporated into vesicles, liposomes or micelles comprising the ligand or activator, and may include ligands or antibodies for targets in the general proximity of the area in which it is desired to activate the relevant PPAR.

It will be appreciated that antibodies may be polyclonal or monoclonal, or may simply comprise the effective or equivalent part thereof (e.g. FAB fragment). Humanised monoclonal antibodies or fragments or equivalents thereof are particularly preferred. Methods used to manufacture humanised monoclonal antibodies are well known in the art.

Liposomes are lipid based vesicles which encapsulate a selected agent which is then introduced into a patient. The liposome is manufactured either from pure phospholipid or a mixture of phospholipid and phosphoglyceride. Typically, liposomes can be manufactured with diameters of less than 200 nm, enabling them to be intravenously injected and to pass through the pulmonary capillary bed. Furthermore the biochemical nature of liposomes confers permeability across blood vessel membranes to gain access to selected tissues. Liposomes have a relatively short half life. So called STEALTH^{R} liposomes-have been developed which comprise liposomes coated with polyethylene glycol (PEG). The PEG treated liposomes have a significantly increased half-life when administered intravenously to a patient.

Formulations may be applied to the patient, as and when desired. In any event, the skilled physician will readily be able to prescribe an effective dose and regimen. The dosage administered will depend on the age, health and weight of the recipient, kind of concurrent treatment, if any, frequency of treatment and the nature of the effect desired. An exemplary systemic daily dosage is about 0.1 mg to about 500 mg. Normally, from about 10 mg to 110 mg daily of the activator or ligand, in one or more dosages per day, is effective to obtain the desired results.

The main reason for the lack of understanding of the cellular basis for the actions of bone anabolic drugs is that there exists, at present, no single in vitro assay which responds to these drugs in an appropriate manner. However, we have now developed a number of assays which, when used in combination with neonatal rat calvarial organ cultures, are able to predict bone anabolic agents.

Thus, according to a further aspect of the present invention there is provided a method for the screening of agents which modulate the activity of PPAR transcription factors comprising:

i providing a culture of bone forming cells;

ii exposing the bone forming cells to an agent capable of modulating the activity of at least one PPAR transcription factor; and

iii monitoring the effect of the agent on the bone forming capacity of the cell culture.

Screens of this type are well known in the art but have not been used to screen for agents which modulate the activity of PPAR transcription factors. For example, these include the calcifying fibroblastic-colony forming unit assay (Scutt A. Bertram P. Bone marrow cells are targets for the anabolic actions of prostaglandin E₂ on bone: Induction of a transition from non-adherent to adherent osteoblast precursors. J. Bone and Mineral Res. 10:474-489, 1995); the non-adherent stromal precursor cell culture screen (Miao D, Scutt A. Non-adherent stromal precursor cells are possible targets for bone anabolic agents. J Bone and Miner. Res. 23:S537, 1998); and the calvarial collagen synthesis screen, for use in monitoring the production of collagen [Chyun Y. S., Raisz L. G., (1984) Stimulation of bone formation by prostaglandin E₂. Prostaglandins 27:97-103].

According to a yet further aspect of the of invention there is provided an agent derived by the screening method according the invention.

In the accompanying Figures, which are purely illustrative and not limiting on the present invention, the effect of certain compounds is shown on alkaline phosphatase activity, calcium uptake and collagen synthesis, respectively, with the final bar showing the cumulative and determinative effect of the compound on bone anabolism:

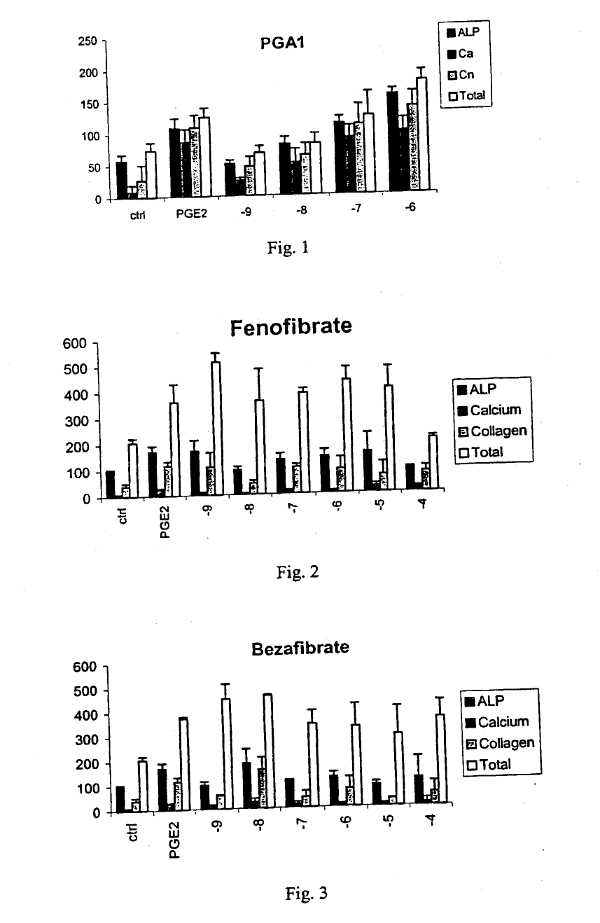

FIG. 1 is the bar chart for PGA₁;

FIG. 2 is the bar chart for fenofibrate;

FIG. 3 is the bar chart for bezafibrate;

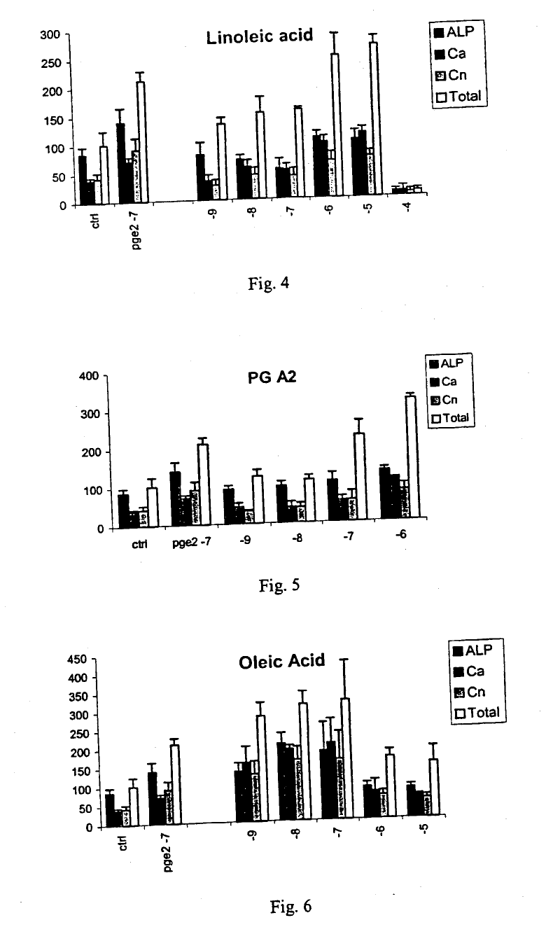

FIG. 4 is the bar chart for linoleic acid;

FIG. 5 is the bar chart for PGA₂;

FIG. 6 is the bar chart for oleic acid; and

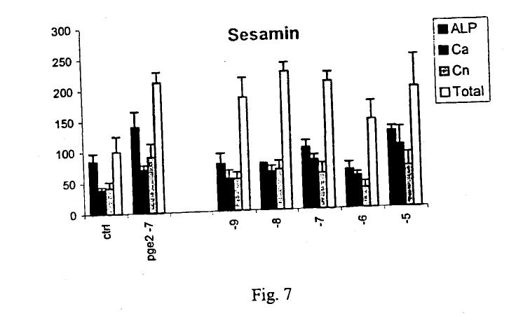

FIG. 7 is the bar chart for sesamin.

The present invention will now be further illustrated in the following, non-limiting Examples.

### PREPARATORY EXAMPLE

Prior to testing the compounds, it was necessary to prepare the assays. Preparation of the materials and the assays used were as follows.

Preparation of Whole Bone Marrow Cells

Whole bone marrow cells (BMC's) were obtained from the tibias and femurs of 125 g male Wistar rats. The bones were removed under aseptic conditions and all soft adherent tissue removed. An end of each bone was removed, a hole made in the opposing end with an 18 gauge syringe needle, and the cells isolated centrifugally [Dobson K. R., et al., Calcif. tissue Int., 65:411-413 (1999)]. The cells were dispersed in 10 ml DMEM (containing 12% FCS, 1×10⁻⁸ M dexamethasone and 50 mg/ml ascorbic acid) by repeated pipetting, and a single-cell suspension achieved by forcefully expelling the cells through a 20 gauge syringe needle. The cells were then used in the protocols described below.

Fibroblastic Colony Forming Unit Cultures

To analyse the numbers of fibroblastic-colony forming units (CFU-f) in either whole BMC or high density non-adherent stromal precursor (NASP) cell cultures, 10⁶ nucleated BMC, or the non-adherent cells from the high density NASP cell cultures, were plated out on 55 cm² petri dishes in DMEM containing; 12% FCS, 1×10⁻⁸ M dexamethasone and 50 μg/ml ascorbic acid. In the case of the CFU-f analysis of whole BMC, the test agents were added once at the beginning of the culture period. In the case of NASP cell cultures, the test agents were added at the beginning of the NASP cell cultures themselves and the CFU-f assay only used to assess the number of CFU-f generated during the NASP cell culture. The medium was changed after 5 days and, thereafter, twice weekly. The cultures were maintained for 18 days, after which the cells were washed with PBS and fixed, by the addition of cold ethanol.

After fixation, the cultures were stained for alkaline phosphatase (APase) positive, calcium positive, collagen positive and total colonies as described by Scutt & Bertram (J. Bone and Mineral Res. 10:474-489, 1995). The cultures were then photographed using a digital camera and the APase positive, calcium positive, collagen positive and total colonies quantitated using Bioimage “Intelligent Quantifier” image analysis software [Dobson K., et al., A cost effective method for the automatic quantitative analysis of fibroblastic-colony forming units with osteoblastic potential. Calcif. Tissue Int. 65:166-172 (1999)].

High-Density NASP Cell Cultures

BMC were cultured at a density of 1.5×10⁶ cells per 2 cm² well in 0.75 ml DMEM containing 12% FCS, 10⁻⁸ M dexamethasone and 50 μg/ml ascorbic acid. Solutions of the agents to be tested were added to the wells and then cultured for 4 days. The numbers of NASP cells present in the supernatant were then quantitated as described above for CFU-f cultures. To do this, the cultures were gently agitated and the supernatants, containing the non-adherent cells, were transferred to 55 cm² petri dishes. 10 ml of DMEM containing 12% FCS, 1×10⁻⁸ M dexamethasone, 50 μg/ml ascorbic acid was added and the cultures maintained further as described above for CFU-f cultures.

Organ Culture of Neonatal Rat Calvariae

One day old rat pups were killed and the calvariae (skull cases) dissected out. The calvariae were then cut along the sagittal suture to give two hemicalvariae per foetus. Each bone was cultured in 2 ml DMEM containing 1 mg/ml BSA, 50 μg/ml ascorbic acid, 60 μg/ml penicillin, and 50 μg/ml streptomycin and 1×10⁻⁸ M dexamethasone in 35 mm tissue culture wells. After 24 h, the medium was replaced with fresh medium, any test agent added, and the tissue cultured for a further 48 h.

Assay of Collagen Synthesis

In this assay, each bone was pulsed with 10 μCi of [³H]proline for 24 h at the end of the culture period. The bones were washed successively in trichloroacetic acid (TCA), acetone, and ether, and then dried. The incorporation of [³H]proline into collagenase-digestible protein (CDP) was determined using purified bacterial collagenase by the method of Peterkofsky B. and Diegelmann R. (Biochemistry, 6: 988-994, 1971) and expressed as dpm.

### EXAMPLE

PGE₂ may be non-enzymatically converted to prostaglandins of the A series (reviewed by Negushi N., et al., Lipid Mediators Cell Signalling 12, 443-448, 1995), and the anabolic activity of PGE₂ may be mediated by these metabolites. Accordingly, PGA₁ was investigated in accordance with the above assays, and was found to produce a positive response in all three of these assays. The results, shown in FIG. 1, were of a magnitude comparable with that produced by PGE₂, indicating bone anabolic activity.

From the results of the tests on other compounds, it can be seen that the fibrate family of compounds all have bone anabolic activity, regardless of the PPAR with which they interact. For example, fenofibrate (FIG. 2) binds PPARα, while bezafibrate (FIG. 3) binds PPARδ. Both have activities superior to that of PGE₂.

As shown above, PGA₁, which is known to be a potent PPARδ agonist, produced a significant dose dependent increase in colony numbers. Methyl palmitate also produced stimulation. Another PPARδ agonist, iloprost, also produced a stimulation comparable with that of PGA₁.

Linoleic acid (FIG. 4), which is known to bind all of the PPAR's, also showed bone anabolic activity.

Other compounds showing useful activity were PGA₂ (FIG. 5), oleic acid (FIG. 6), and sesamin (FIG. 7). In general, compounds showing useful activity were taken as those having an equivalent activity to that of PGE₂, although it will be appreciated that any compound having an activity over that of a control with no compound, is good, in comparison with the art.

Previously, the most active bone anabolic agent was PGE₂. However, because the PGE₂ receptors are ubiquitous, its use gives rise to many serious complications, including vomiting, diarrhoea, spontaneous abortion and most seriously circulatory collapse. However, the PGE₂ metabolite PGA₁ exhibits a level of activity at least as good as that of PGE₂. PGA₁ does not bind to a cell membrane receptor and, so, is unlikely to give rise to the side effects seen with PGE₂.

As noted above, there is no single assay that can reliably report on bone anabolic activity. Individual assays can identify certain putative bone anabolic agents but as there are many bone anabolic agents which act by a number of mechanisms, many remain unidentified. By using the CFU-f and the NASP cell assays in combination with neonatal rat calvarial organ cultures, bone anabolic agents can be reliably identified as the false negatives are reduced to a minimum.

1. Use of a compound which is an activator or ligand of a peroxisome proliferator-activated receptor other than PPARγ, or pharmaceutically acceptable derivative of said activator or ligand, in the manufacture of a medicament for the treatment or prophylaxis of bone disease.

2. Use of an activator according to claim 1, wherein the activator is a pan-activator.

3. Use according to claim 2, wherein the activator is linoleic acid, linolenic acid or arachidonic acid.

4. Use according to claim 1, wherein the ligand is an agonist.

5. Use according to claim 4, wherein the ligand is an agonist of PPARα or PPARδ.

6. Use according to any preceding claim wherein the substance has equal or greater bone anabolic activity than PGE₂.

7. Use according to claim 1 wherein the substance is an antagonist.

8. Use according to claim 7, wherein the bone disease is Paget's disease.

9. Use according to claim 1, wherein the substance is a fibrate,

10. Use according to claim 9, wherein the substance is fenofibrate or bezafibrate.

11. Use according to claim 1, wherein the substance is a N-(2-benzoylphenyl)-L-tyrosine derivative.

12. Use according to claim 1, wherein the substance is PGA₁, PGA₂ or sesamin

13. Use according to claim 1, wherein the substance is: 3-{4-[2-(2-benzoxazolylmethylamino)ethoxy]benzene}-2-(2S)-(2,2,2-trifluoroethoxy)propanoic acid; docosahexaenoic acid; LY171883; linoleic acid; oleic acid; palmitic acid; clofibrate; eicosatetraenoic acid; 8(S)-hydroxy-6,8,11,14-eicosatetraenoic acid; methyl palmitate; Wy-14643 ([4-chloro-6-(2,3-xylidino)-2-pyrimidinylthio]acetic acid); nafenopin {2-methyl-2[p-(1,2,3,4-tetrahydro-1-naphthyl)phenoxy]propionic acid}; clofibric acid [2-([p]-chlorophenoxy)-2-methylpropionicacid]; MK-571 ((+-)-3-[({3-[2-(7-chloro-2 quinolinyl)ethenyl]-phenyl} {[3-(dimethylamino)-3-oxopropyl]thio}methyl)-(thio) (propanoic acid); PGJ(2)[prostaglandin J₂]; Δ(12)PGJ(2) [Δ(12)prostaglandin J₂]; 15-deoxy-Δ(12,14)-PGJ(2) [15-deoxy-Δ(12,14)-prostaglandin J₂]; PD19559; conjugated linoleic acid; carbaprostacyclin; 9-hydroxyoctadecadienoic acid; KRP-297; Iloprost; L783483; petroselinic acid; elaidic acid; erucic acids, linolenic acid; L165461; L796449; L165041; GW2433; GW1929; GW2331; 2 bromopalmitate; heptyl-4-yn-VPA (heptyl-4-yn-valproic acid); hexyl-4-yn-VPA (hexyl-4-yn-valproic acid); methyl palmitate; 4-[3-(2-propyl-3-hydroxy-4-acetylphenoxy)propyloxy]-phenoxyacetic acid; 3-chloro-4-{3-[2-propyl-3-hydroxy-4-(1-hydroxliminopropyl)-phenoxy]propylthio}phenylacetic acid; 3-chloro-4-[3-(3-ethyl-7-propyl-6-benz [4,5]-isoxazoloxy)propylthio]phenyl acetic acid; 3-chloro-4-[3-(2-propyl-3-trifluoromethyl-6-benz-[4,5]-isoxazoloxy)propylthio]phenyl acetic acid; 4-(2-acetyl-6-hydroxyundecyl)cinnamic acid; 3-chloro-4-[3-(3-phenyl-7-propylbenzofuran-6-yloxy)propylthio]phenylacetic acid; or 3-propyl-4-[3-(3-trifluoromethly-7-propyl-6-benz[4,5]-isoxazoloxy)propylthio]phenyl acetic acid.

14. Use of a derivative according to any preceding claim, which is a pro-drug, salt or ester.

15. Use according to any preceding claim, wherein the bone disease is: osteoporosis; Paget's disease; osteogenesis imperfecta; hypophosphatasia; hyperparathyroidism; deafness; orthodontic abnormalities; or cancers which result in hypercalcaemia, especially myeloma.

16. A method for the screening of agents which modulate the activity of PPAR transcription factors comprising:
i providing a culture of bone forming cells;
ii exposing the bone forming cells to an agent capable of modulating the activity of at least one PPAR transcription factor; and
iii monitoring the effect of the agent on the bone forming capacity of the cell culture.
